(19) United States
(12) Patent Application Publication
(10) Pub. No.: US 2022/0211091 A1
(43) Pub. Date: Jul. 7, 2022

(54) COMPOSITIONS COMPRISING CURCUMIN AND COENZYME Q10

(71) Applicant: Indena S.P.A., Milano (IT)

(72) Inventors: Massimo Ronchi, Milano (IT); Sabrina Arpini, Milano (IT); Giacomo Mombelli, Milano (IT); Giuseppe Ramaschi, Milano (IT)

(73) Assignee: Indena S.P.A., Milano (IT)

(21) Appl. No.: 17/439,829

(22) PCT Filed: Mar. 9, 2020
(86) PCT No.: PCT/EP2020/056166

§ 371 Date: Sep. 16, 2021

(30) Foreign Application Priority Data
Mar. 18, 2019 (IT) 102019000003907

(51) Int. Cl. A23L 33/105; A23L 33/12; A61K 31/12; A61K 36/9066; A61K 9/14

(52) U.S. Cl. CPC A23L 33/105; A23L 33/12; A61K 9/146; A61K 36/9066; A61K 31/12

(57) ABSTRACT

The invention relates to compositions in the form of solid dispersions, comprising: a) curcumin or an extract containing it; b) coenzyme Q10; c) a phospholipid, and d) a pharmaceutically or nutraceutically acceptable carrier. The invention also relates to a process for the preparation of the compositions and the use of the compositions to prepare pharmaceutical or nutraceutical formulations for use in enhancing energy metabolism and the cognitive functions and improving the cardiovascular function and the quality of aging.

### FIELD OF INVENTION

The present invention relates to compositions in the form of solid powdered dispersions useful to prepare pharmaceutical or nutraceutical compositions, in particular dispersions comprising curcumin or turmeric extracts and coenzyme Q10 and/or ubiquinol and/or ubiquinol salts.

### BACKGROUND TO THE INVENTION

Extracts of turmeric (Curcuma longa L.), in particular turmeric rhizome extracts, have long been known for their therapeutic potential against various pathological conditions, especially those wherein the inflammatory component is significant. In fact, curcumin [(1E,6E)-1,7-bis-(4-hydroxy-3-methoxyphenyl)-hepta-1,6-diene-3,5-dione], the main constituent of turmeric extracts, has a high antioxidant power and the ability to inhibit cyclooxygenase 2.

However, curcumin administration is affected by the problem of low bioavailability, as curcumin undergoes extensive conjugation (glucuronidation and sulfation) and reduction metabolism, which reduces the curcumin concentration in the plasma and the target tissues.

WO2007/101551 (Indena S.p.A.) discloses solid compositions based on curcumin or extracts containing curcumin in combination with phospholipids and a process for the preparation of said combinations. Said combinations, obtainable by dissolving curcumin or extracts containing it in ethanol and subsequent heating to reflux in the presence of phospholipids, in a phospholipid:curcumin (or extracts containing it) weight ratio ranging from 10:1 to 1:1, provide greater bioavailability of curcumin.

In view of its therapeutic potential, curcumin has been combined with other active ingredients of natural origin in such a way as to supplement or increase their activity. There is therefore still a need for compositions containing curcumin in combination with other active ingredients. For example, the combination with other antioxidant ingredients would produce formulations with greater antioxidant efficacy.

Coenzyme Q10 (or ubiquinone or ubidecarenone), an endogenous lipophilic substance present in numerous eukaryotic cells, mainly in the mitochondria, participates as cofactor in oxidation-reduction reactions of the electron transport chain for ATP production. Coenzyme Q10 exists in three states of oxidation: the fully oxidised form (ubiquinone), the partly reduced form (semiquinone) and the fully reduced form (ubiquinol), and the three forms coexist in a physiological balance in the human body. As coenzyme Q10 is introduced with the diet, especially in foods of animal origin, a deficiency of said cofactor is rare. However, there are some physiological conditions wherein it is necessary or advantageous to supplement the administration of coenzyme Q10, for example if it is necessary to promote energy metabolism, or treat disorders associated with aging, periodontal disease, fatigue, memory disorders, coronary disease, elevated blood pressure or immune deficiency.

Moreover, coenzyme Q10 supplementation can be advantageous to elderly people, to counteract cognitive decline.

It would therefore be advantageous to combine curcumin or extracts containing it with coenzyme Q10, in such a way as to obtain compositions that combine the biological properties of both active ingredients.

However, coenzyme Q10 is characterised by low oral bioavailability. In fact, coenzyme Q10 has a relatively high molecular weight, and is available as a highly lipophilic crystalline powder. Also for coenzyme Q10, the problem of limited bioavailability can be advantageously overcome by preparing solid dispersions with phospholipids, according to the teachings of WO2016/198576 (Indena S.p.A.).

It is also known that phospholipids, due to their physicochemical characteristics (stickiness, tendency to soften), can adversely affect the formulation processes of solid compositions containing them, and that a sufficiently large amount of processing aids is required to obtain dosage forms with acceptable technological properties, especially as regards disintegration time. The development of a dosage form characterised by high patient compliance (acceptable size, once-daily administration), which separately combines effective amounts of two or more compositions wherein the active ingredients are separately combined with phospholipids, is therefore problematic.

### DETAILED DESCRIPTION OF THE INVENTION

It has now been found that compositions in the form of solid powdered dispersions comprising both curcumin, or extracts containing it, and coenzyme Q10 combined with phospholipids, can be prepared without using excessive amounts of phospholipids and excipients; in particular, it has been found that, the amount of each active ingredient contained in separate compositions being equal, the compositions according to the invention contain a significantly lower total amount of phospholipid and other excipients than the sum of the phospholipid and excipient contained in the separate compositions.

It has also been found that despite the reduced content of phospholipid and other excipients, the solubility of the curcumin is unaffected.

The present invention therefore relates to compositions in the form of solid dispersions, typically solid powdered dispersions, comprising:

a) curcumin or an extract containing it;

b) coenzyme Q10;

c) a phospholipid, and

d) a pharmaceutically or nutraceutically acceptable carrier.

For the purposes of the present invention:
the term “curcumin” identifies [(1E,6E)-1,7-bis-(4-hydroxy-3-methoxyphenyl)-hepta-1,6-diene-3,5-dione, of natural or synthetic origin. The term “extracts containing curcumin” indicates extracts of Curcuma longa L. rhizomes wherein the curcuminoid content (curcumin, demethoxycurcumin and bisdemethoxycurcumin) is preferably equal to or greater than 95%;
the term “coenzyme Q10” identifies the fully oxidised form (ubiquinone), the partly reduced form (semiquinone) and the fully reduced form (ubiquinol), and pharmaceutically acceptable salts of the latter substances;
the term “phospholipid” identifies a group of substances selected from the group of lecithins obtained from soya, sunflower or another source consisting of phosphatidylcholine, phosphatidyl serine, phosphatidyl ethanolamine or mixtures thereof, wherein the acyl groups, which can be the same or different, are mainly derivatives of palmitic, stearic, oleic, linoleic or linolenic acids; the phospholipid is preferably selected from soya lecithin and sunflower lecithin;
the term “pharmaceutically or nutraceutically acceptable carrier” indicates one or more solid compounds which are not biologically active, and are soluble or insoluble in water. Examples of water-soluble carriers comprise mono- and disaccharides (such as sucrose, fructose and maltodextrins); polyalcohols (such as mannitol, sorbitol and xylitol); oligo- and polysaccharides (such as dextrans and pullulans), and hydroxypropyl methylcellulose. In a preferred embodiment, the water-soluble carrier is a maltodextrin, hydroxypropyl methylcellulose or a combination thereof. Examples of water-insoluble carriers are microcrystalline cellulose, silicon dioxide and calcium phosphate. In a preferred embodiment, the water-insoluble carrier is silicon dioxide.

According to a particularly preferred embodiment, the pharmaceutically acceptable carrier consists of a mixture of maltodextrin, hydroxypropyl methylcellulose and silicon dioxide.

The compositions in the form of a solid dispersion according to the invention are obtainable by a process comprising the following steps:
a) preparing a solution of curcumin, or of an extract containing curcumin, and coenzyme Q10 in an organic solvent;
b) preparing a solution or suspension of a phospholipid in the same solvent as used for step a);
c) mixing the solution obtained in step a) with the solution or suspension obtained in step b) to obtain a solution or suspension comprising curcumin or an extract containing curcumin, coenzyme Q10 and a phospholipid;
d) adding a pharmaceutically or nutraceutically acceptable carrier to the solution or suspension obtained in step c) to obtain a solution or suspension comprising curcumin or an extract containing curcumin, coenzyme Q10, a phospholipid and a carrier;
e) removing the solvent.

For the purposes of the present invention, the term “solution” indicates a liquid composition which appears clear on visual inspection; the term “suspension” indicates a liquid composition which, on visual inspection, contains suspended particles and appears opaque and cloudy, but still homogeneous.

The organic solvent is preferably selected from ethyl alcohol, ethyl acetate and acetone; according to a preferred embodiment, the organic solvent is ethanol. A volume of solvent ranging from 5 to 15 volumes compared with the weight of the active ingredients is generally used in step a); a volume of solvent ranging from 5 to 10 volumes compared with the weight of the phospholipid is used in step b).

The weight ratio of the phospholipid to the total amount of active ingredients ranges from 0.5 to 1.5; preferably, the ratio of phospholipid to the total amount of active ingredients is 1.

Typically, the weight ratio (R) of the total amount of phospholipid+carrier to the total amount of active ingredients [R=(phospholipid+carrier)/total amount of active ingredients] ranges from 2.5(R=2.5/1) to 1 (R=1/1), preferably from 2.5 (R=2.5/1) to 2 (R=2/1).

Preferably, steps a) and b) are conducted by heating the solution or suspension to a temperature ranging from 40° C. 2 80° C., preferably from 50° C. 2 65° C., and maintaining it under stirring.

Step e) is preferably performed by low-pressure evaporation or spray-drying, until a solvent residue preferably lower than the limits specified in the part of ICH Guideline Q3C(R6) on residual solvents relating to class 3 solvents is obtained.

Typically, the composition undergoes final sieving to obtain a composition with a particle size ranging from 50 to 1000 μm, preferably from 100 to 500 μm.

Finally, the compositions according to the invention can be formulated, optionally by adding further pharmaceutically or nutraceutically acceptable carriers or excipients in pharmaceutical/nutraceutical formulations suitable for single administration, such as granulates, tablets, capsules, etc., according to methods and techniques known in the industry, for example as described in Remington: “The Science and Practice of Pharmacy” 22nd edition, Pharmaceutical Press, 2013.

The compositions according to the invention and the formulations obtained from them are useful for administration in all conditions wherein the antioxidant/anti-inflammatory effect of curcumin needs to be combined with the antioxidant effect of coenzyme Q10.

In addition to the permeability of the intestinal mucosa, the concentration of an active ingredient in the intestinal fluids, which depends on its solubility in said fluids, plays a crucial part in determining the rate and extent of its absorption (A review of the solubility in human intestinal fluids: implication for the prediction of oral absorption. Patric Augustijns et al.; Eur. J. Pharm. Sci., 57 (2014) 322-332).

For those reasons, assays were conducted in fed-state and fasted-state simulated intestinal fluids. Said assays demonstrated that although the compositions according to the invention contain a smaller amount of phospholipids and carrier than that which would be obtained by combining compositions containing only a turmeric extract as active ingredient and compositions containing only coenzyme Q10 as active ingredient, the solubility of curcuminoids, in particular curcumin, is greater than that of compositions containing only a turmeric extract as active ingredient.

The invention is described in greater detail in the following experimental part.

### EXPERIMENTAL PART

Materials

A commercial turmeric extract with an HPLC assay value in curcuminoids exceeding 95% was used.

The coenzyme Q10 was obtained from Shenzhou Biology & Technology Co., Ltd or Kaneka Nutrients.

The soya or sunflower lecithin was obtained from Cargill or Novastell.

The tests of dissolution in fasting-state and fed-state simulated intestinal fluid were conducted with fluids obtained from Biorelevant.com Ltd, according to the method recommended by the supplier.

### EXAMPLES OF FORMULATIONS

### Example 1—Composition (C-1) (Composition According to the Invention)

A composition according to the invention comprising the ingredients listed in Table 1 below:

TABLE 1

| | % weight of the total | |
|---|---|---|
| Ingredients | composition | mg/dosage unit |
| Turmeric extract | 20.0 | 100.0 |
| Coenzyme Q₁₀ | 10.0 | 50.0 |
| Sunflower lecithin | 30.0 | 150.0 |
| Maltodextrin | 30.0 | 150.0 |
| Hydroxypropyl | 8.0 | 40.0 |
| methylcellulose | | |
| Silicon dioxide | 2.0 | 10.0 |
| TOTAL | 100.0 | 500.0 |

was prepared by the following process:
a-1) the turmeric extract and coenzyme Q10 were solubilised in 10 volumes of ethyl alcohol, and the solution was heated to 55-60° C.;
b-1) the sunflower lecithin was dispersed in 5 volumes of ethyl alcohol at 55-60° C.;
c-1) the solution obtained in step a-1) was combined with the dispersion obtained in step b-1);
d-1) maltodextrin and hydroxypropyl methylcellulose were added under stirring to the dispersion obtained in step c-1), and the stirring and temperature were maintained for 5 minutes;

The mixture obtained in step d-1) was transferred to a rotary evaporator, and the solvent was removed under the following experimental conditions: temperature 55° C., flask rotation 80-100 rpm, and continuous vacuum 180 mbars.

After about 45 minutes the partly dried mass was transferred to the drying tray of a stove under vacuum, and drying continued for about 4 hours at 55° C.

The resulting product was calibrated on a 10-mesh screen and dried under vacuum at 55° C. for about 2 hours, until an ethyl alcohol residue <5000 ppm was obtained, after which silicon dioxide was added and the resulting solid dispersion was then ground in a mill equipped with a 2 mm grid.

### Example 2—Reference Composition (Cᵣ-1) Containing Turmeric Extract Only

A reference composition containing only turmeric extract as active ingredient, having the composition reported in Table 2 below:

TABLE 2

| Ingredients | % | mg/dosage unit |
|---|---|---|
| Turmeric extract | 20.0 | 100.0 |
| Sunflower lecithin | 40.0 | 200.0 |
| Microcrystalline cellulose | 40.0 | 200.0 |
| TOTAL | 100.0 | 500.0 |

was prepared by the following process:
a-2) the turmeric extract was solubilised in 10 volumes of ethyl alcohol, and the solution was heated to about 70° C.;
b-2) the sunflower lecithin was dispersed in 5 volumes of ethyl alcohol to about 70° C.;
c-2) the solution obtained in step a-2) was combined with the dispersion obtained in step b-2);
d-2) microcrystalline cellulose was added under stirring to the dispersion obtained in step c-2), and the stirring and temperature were maintained for 5 minutes;

The mixture obtained in step d-2) was transferred to the flask of a rotary evaporator, and the solvent was removed under the following experimental conditions: temperature 60° C., flask rotation 80-100 rpm, and continuous vacuum 180 mbars.

After about 45 minutes the partly dried mass was transferred to the drying tray of a stove under vacuum, and drying continued for about 4 hours at 60° C.

The product was calibrated on a 10-mesh screen and dried under vacuum at 60° C. for about 2 hours, until an ethyl alcohol residue <5000 ppm was obtained.

The resulting composition (Cᵣ-1) was ground in a mill equipped with 2 mm grid.

### Example 3—Reference Composition (Cᵣ-2) Containing Coenzyme Q10 Only

A reference composition containing only coenzyme Q10 as active ingredient, having the composition reported in Table 3 below:

TABLE 3

| Ingredients | % | mg/dosage unit |
|---|---|---|
| Coenzyme Q₁₀ | 20.0 | 50.0 |
| Sunflower lecithin | 20.0 | 50.0 |
| Maltodextrin | 48.0 | 120.0 |
| Hydroxypropyl | 10.0 | 25.0 |
| methylcellulose | | |
| Silicon dioxide | 2.0 | 5.0 |
| TOTAL | 100.0 | 250.0 |

was prepared by the following process:

a-3) the coenzyme Q10 was dissolved in 10 volumes of ethyl acetate, and the solution was heated to 55-60° C.;

b-3) the sunflower lecithin was partly dissolved in 5 volumes of ethyl acetate at about 55-60° C.

c-3) the solution obtained in step a-3) and the dispersion obtained in step b-3) were combined;

d-3) maltodextrin and hydroxypropyl methylcellulose were added under stirring to the mixture obtained in step c-3), and the stirring and temperature were maintained for 5 minutes.

The mixture obtained in step d-3) was transferred to the flask of a rotary evaporator, and the solvent was removed under the following experimental conditions: temperature 55° C., flask rotation 80-100 rpm, and continuous vacuum 180 mbars.

After about 45 minutes the partly dried mass was transferred to the drying tray of a stove under vacuum, and drying continued for about 4 hours at 55° C.

The resulting product was calibrated on a 10-mesh screen and dried under vacuum at 55° C. for about 2 hours, until an ethyl acetate residue <5000 ppm was obtained, after which silicon dioxide was added and the resulting solid dispersion was then ground in a mill equipped with a 2 mm grid.

Table 4 below shows a weight comparison between a theoretical composition (single dose) that would be obtained by totaling the weights of the ingredients of the two reference compositions and the composition (C-1) according to the invention. It will be observed that whereas the total weight of a single dose of theoretical composition amounts to 750 mg, that of the composition according to the invention amounts to 500 mg, but contains the same amount of the two active ingredients as the two reference compositions. It will also be seen that the sunflower lecithin content in composition (C-1) is equal to that of the total weight of the active ingredients, whereas in the theoretical composition the lecithin/active ingredient weight ratio is greater than 1.5.

TABLE 4

| | | | Theoretical | |
|---|---|---|---|---|
| | | | composition | |
| | | | resulting from | |
| | | | combination of | |
| | Compo- | Compo- | the reference | Compo- |
| | sition | sition | compositions | sition |
| | (Cᵣ-1) | (Cᵣ-2) | (Cᵣ-1) and | (C-1) |
| Ingredients | (mg/dose) | (mg/dose) | (Cᵣ-2) (mg/dose) | (mg/dose) |
| 95% curcumin | 100 | — | 100 | 100 |
| Coenzyme Q₁₀ | — | 50 | 50 | 50 |
| Sunflower | 200 | 50 | 250 | 150 |
| lecithin | | | | |
| Microcrystalline | 200 | — | 200 | — |
| cellulose | | | | |
| Maltodextrin | — | 120 | 120 | 150 |
| Hydroxypropyl | — | 25 | 25 | 40 |
| methylcellulose | | | | |
| Silicon dioxide | — | 5 | 5 | 10 |
| Total weight of | 100 | 50 | 150 | 150 |
| active | | | | |
| ingredients | | | | |
| Total weight of | 400 | 200 | 600 | 350 |
| carriers | | | | |
| Total weight of | 500 | 250 | 750 | 500 |
| dosage unit | | | | |

Solubility Test in Simulated Biological Fluids

The Tables below show the results of the solubility tests in fasted-state and fed-state simulated intestinal fluids, in particular the solubility values of the curcuminoids, comparing a turmeric extract “as is”, reference composition (Cᵣ-1) and composition C-1 according to the invention. As demonstrated by the results, although the compositions according to the invention contain a smaller amount of phospholipids and carrier than that which would be obtained by combining formulations containing only a turmeric extract as active ingredient, and formulations containing only coenzyme Q10 as active ingredient, the solubility of curcuminoids, in particular curcumin, is greater than that of formulations containing only a turmeric extract as active ingredient.

TABLE 5

| | | Solubility | Solubility |
|---|---|---|---|
| | Solubility of | (mg/ml) of | (mg/ml) of |
| Fasted-state | curcuminoids | curcuminoids | curcuminoids |
| simulated | in turmeric | in reference | in compos- |
| intestinal fluid | extract | composition | ition |
| (FaSSIF) - pH 6.5 | (mg/ml) | (Cᵣ-1) | (C-1) |
| Bisdemethoxy- | 0.001 | 0.060 | 0.029 |
| curcumin | | | |
| Demethoxy- | 0.001 | 0.207 | 0.128 |
| curcumin | | | |
| Curcumin | 0.003 | 0.084 | 0.205 |
| Total curcuminoids | 0.005 | 0.351 | 0.362 |

TABLE 6

| | | Solubility | Solubility |
|---|---|---|---|
| | Solubility of | (mg/ml) of | (mg/ml) of |
| Fed-state | curcuminoids | curcuminoids | curcuminoids |
| simulated | in turmeric | in reference | in compo- |
| intestinal fluid | extract | composition | sition |
| (FeSSIF) - pH 5.0 | (mg/ml) | (Cᵣ-1) | (C-1) |
| Bisdemethoxy- | 0.002 | 0.128 | 0.061 |
| curcumin | | | |
| Demethoxy- | 0.006 | 0.443 | 0.280 |
| curcumin | | | |
| Curcumin | 0.019 | 0.184 | 0.464 |
| Total curcuminoids | 0.027 | 0.755 | 0.805 |

1. Compositions in the form of solid dispersions, comprising:
a) curcumin or an extract containing it;
b) coenzyme Q10;
c) a phospholipid; and
d) a pharmaceutically or nutraceutically acceptable carrier.

2. Compositions according to claim 1 wherein ingredient b) is coenzyme Q10 in oxidised, partially reduced (semiquinone) or fully reduced (ubiquinol) forms and the pharmaceutically acceptable salts thereof.

3. Compositions according to claim 1 wherein the phospholipid is selected from phosphatidylcholine, phosphatidyl serine, phosphatidyl ethanolamine and mixtures thereof.

4. Compositions according to claim 1 wherein the phospholipid is selected from soya and sunflower lecithins.

5. Compositions according to claim 1 wherein the weight ratio of phospholipid to the total amount of the active ingredients b) and c) ranges from 0.5 to 1.5.

6. Compositions according to claim 1 wherein the weight ratio of the total amount of the phospholipid and carrier to the total amount of active ingredients amounts ranges from 2.5 to 2.

7. A process for preparation of the compositions of claim 1, said method comprising:
a) preparing a solution of curcumin or an extract containing curcumin and coenzyme Q10 in an organic solvent;
b) preparing a solution or suspension of a phospholipid in the solvent used in step a);
c) mixing the solution of step a) with the solution or suspension of step b) to obtain a solution or suspension comprising curcumin or an extract containing curcumin, coenzyme Q10 and a phospholipid;
d) adding a carrier to the solution or suspension of step c) to obtain a solution or suspension comprising curcumin or an extract containing curcumin, coenzyme Q10, a phospholipid and a carrier;
e) removing the solvent.

8. Process according to claim 7 wherein the organic solvent is selected from ethanol, ethyl acetate and acetone.

9. Pharmaceutical or nutraceutical formulations comprising the compositions of claim 1.

10. (canceled)

11. Compositions according to claim 5 wherein the weight ratio of phospholipid to the total amount of the active ingredients b) and c) is 1.
